(19) United States
(12) Patent Application Publication
(10) Pub. No.: US 2011/0300608 A1
(43) Pub. Date: Dec. 8, 2011

(54) NUCLEIC ACID ISOLATION IN PRESERVED WHOLE BLOOD

(75) Inventors: Wayne L. Ryan, Omaha, NE (US); Kate Chao-Wei Chan, Indianapolis, IN (US)

(73) Assignee: Streck, Inc., LaVista, NE (US)

(21) Appl. No.: 12/678,190

(22) PCT Filed: Sep. 17, 2008
(86) PCT No.: PCT/US08/76590

§ 371 Date: Aug. 29, 2011

Related U.S. Application Data
(60) Provisional application No. 60/974,115, filed on Sep. 21, 2007.

(51) Int. Cl. C07H 1/08; C07H 21/04; C12S 3/20

(52) U.S. Cl. 435/270

(57) ABSTRACT

A method for isolating nucleic acids is disclosed, wherein a sample having nucleic acid containing starting material is fixed, lysed, and treated to remove unwanted contaminants. The initial fixing of the sample aids in maintaining the structure and integrity of the isolated DNA and reduces the incidence of end product contaminants and DNA shearing.

### CLAIM OF BENEFIT OF FILING DATE

The present application claims the benefit of the filing date of U.S. Provisional Application Ser. No. 60/974,115 (filed Sep. 21, 2007), incorporated by reference herein.

### FIELD OF THE INVENTION

This invention relates to DNA isolation from a biological sample and more particularly to the isolation of DNA from whole blood having been fixed and preserved.

### BACKGROUND OF THE INVENTION

The isolation of DNA is a necessary step in many diagnostic testing procedures. In general, DNA isolation uses a series of extraction and washing steps which often result in DNA shearing and the failure to remove unwanted materials from the DNA sample. A contaminated DNA sample makes it difficult if not impossible to use the sample as a diagnostic tool. A number of patent documents address such processes for the isolation of DNA and RNA. See, generally, U.S. Pat. Nos. 7,173,124; 6,914,137; 6,548,256; 5,945,515; and 5,898,071 all incorporated by reference herein. Notwithstanding the above, there remains a need for DNA isolation methods that can be performed in an efficient and inexpensive manner while maintaining the integrity of the DNA and eliminating the shearing and contaminants often associated with traditional methods of isolation.

The most common method for isolating nucleic acids involves lysing the sample containing the DNA, extracting the mixture with an organic solvent and precipitating the DNA through the addition of alcohol. This method is time consuming and involves the use of hazardous materials in that it commonly requires the use of phenol or other toxic organic solvents.

To avoid the use of hazardous materials, another method involves lysing the sample containing the DNA with a chaotropic substance such as urea or guanidinium chloride and combining the sample with a DNA binding solid phase. The DNA binds to the solid phase and any remaining unwanted components or impurities are washed away. While less time consuming, this process results in unwanted contaminants and often removal of sections of the DNA, or DNA shearing.

Further methods include mixing an initial sample with a detergent substance which acts to separate unwanted lipids and proteins from the DNA. The unwanted contaminants are removed and the DNA is extracted using a salt. While again avoiding the use of toxic chemicals, this method usually results in undesired DNA shearing and contaminants.

A blood or tissue sample fixed with certain fixatives is disclosed in U.S. Pat. Nos. 5,196,182; 5,260,048; 5,459,073; 5,460,797; 5,811,099; and 5,849,517, each incorporated herein by reference. A blood or tissue sample may also be collected and fixed in a specific type of tube, such as that disclosed in U.S. application Ser. No. 10/605,669, incorporated herein by reference.

The present invention addresses the need for an efficient and consistent method of DNA extraction by providing an improved method for the isolation of nucleic acids from a biological sample including the initial step of fixing the biological sample in order to maintain the structural integrity and purity of the isolated DNA.

### SUMMARY OF THE INVENTION

In a first aspect, the present invention contemplates a method for isolating DNA comprising: providing a tube containing an anticoagulant agent and a fixative agent; suspending a sample in the fixative agent; contacting the sample with an erythrocyte lysis buffer; contacting the sample with a nucleus lysis buffer; contacting the sample with proteinase K; and contacting the sample with ethanol.

This aspect may be further characterized by one or any combination of the following features: the fixative agent is selected from the group consisting of diazolidinyl urea, imidazolidinyl urea, dimethoylol-5,5-dimethylhydantoin, dimethylol urea, 2-bromo-2.-nitropropane-1,3-diol, oxazolidines, sodium hydroxymethyl glycinate, hydroxymethoxymethyl-1-1aza-3,7-dioxabicyclo[3.3.0]octane, 5-hydroxymethyl-1-1aza-3,7dioxabicyclo[3.3.0]octane, 5-hydroxypoly[methyleneoxy]methyl-1-1aza-3,7dioxabicyclo [3.3.0]octane, quaternary adamantine and combinations thereof, the erythrocyte lysis buffer includes ammonium chloride, ammonium bicarbonate, and a chelating agent, wherein the chelating agent is EDTA, the nucleus lysis buffer includes ingredients selected from the group consisting of a chelating agent, a buffer, an anionic surfactant, a polysorbate surfactant, a non-ionic surfactant, and a chaotrope, the nucleus lysis buffer includes a buffer, a chelating agent and an anionic surfactant and the buffer is tris-HCL.

In another aspect, the present invention contemplates a method for isolating DNA comprising: providing a tube containing an anticoagulant agent and a fixative agent selected from the group consisting of diazolidinyl urea, imidazolidinyl urea, and combinations thereof; suspending a sample in the fixative agent; contacting the sample with ammonium chloride, ammonium bicarbonate, and EDTA; contacting the sample with a nucleus lysis buffer wherein the nucleus lysis buffer contains a buffer, a chelating agent and an anionic surfactant; contacting the sample with proteinase K; and contacting the sample with ethanol.

This aspect may be further characterized by one or any combination of the following features: the buffer is tris-HCl, the chelating agent is EDTA and the anionic surfactant is sodium dodecyl sulfate, the fixed sample is transferred to a remote location prior to a cell lysis processing step and the isolated DNA is analyzed and resulting data is provided to an initial sample draw location, the remote location, an additional location, or any combination thereof.

In a further aspect, the present invention contemplates a method of DNA isolation and analysis comprising: providing a tube containing an anticoagulant agent and a fixative agent selected from the group consisting of diazolidinyl urea, imidazolidinyl urea, and combinations thereof; extracting a sample from a patient at a sample extraction site; suspending the sample in the fixative agent; transporting the fixed sample to a remote location; contacting the sample with ammonium chloride, ammonium bicarbonate, and EDTA; contacting the sample with a nucleus lysis buffer wherein the nucleus lysis buffer contains a buffer, a chelating agent and an anionic surfactant; contacting the sample with proteinase K; contacting the sample with ethanol; analyzing any resulting isolated DNA; providing data regarding the resulting isolated DNA to the sample extraction site, the remote location, an additional site, or any combination thereof.

In a further aspect, the present invention contemplates a method for isolating DNA comprising: providing a tube containing an anticoagulant agent and a fixative agent; suspending a sample in the fixative agent; contacting the sample with an erythrocyte lysis buffer; contacting the sample with a nucleus lysis buffer; contacting the sample with proteinase K; and contacting the sample with ethanol.

This aspect may be further characterized by one or any combination of the following features: the fixative agent is selected from the group consisting of diazolidinyl urea, imidazolidinyl urea, dimethoylol-5,5-dimethylhydantoin, dimethylol urea, 2-bromo-2.-nitropropane-1,3-diol, oxazolidines, sodium hydroxymethyl glycinate, 5-hydroxymethoxymethyl-1-1aza-3,7-dioxabicyclo[3.3.0]octane, 5-hydroxymethyl-1-1aza-3,7dioxabicyclo[3.3.0]octane, 5-hydroxypoly[methyleneoxy]methyl-1-1aza-3,7dioxabicyclo [3.3.0]octane, quaternary adamantine and combinations thereof, the fixative agent is diazolidinyl urea, the fixative agent is imidazolidinyl urea, the erythrocyte lysis buffer includes ammonium chloride, ammonium bicarbonate, and a chelating agent, the chelating agent is EDTA, the nucleus lysis buffer includes ingredients selected from the group consisting of a chelating agent, a buffer, an anionic surfactant, a polysorbate surfactant, a non-ionic surfactant, and a chaotrope, the nucleus lysis buffer includes a buffer, a chelating agent, a polysorbate surfactant, a non-ionic surfactant and a chaotrope, the nucleus lysis buffer includes a buffer, a chelating agent and an anionic surfactant, the chelating agent is EDTA, the buffer is tris-HCL, the chelating agent is EDTA and the anionic surfactant is sodium dodecyl sulfate, one or more samples are extracted from one or more patients at a sample extraction site, the sample suspended in the fixative agent is transferred to a remote location prior to a cell lysis processing step or isolated DNA is analyzed and resulting data is provided to the sample extraction site, the remote location, an additional location, or any combination thereof.

### BRIEF DESCRIPTION OF THE DRAWINGS

FIG. 1 is a flow diagram illustrating an example protocol for a DNA isolation method.

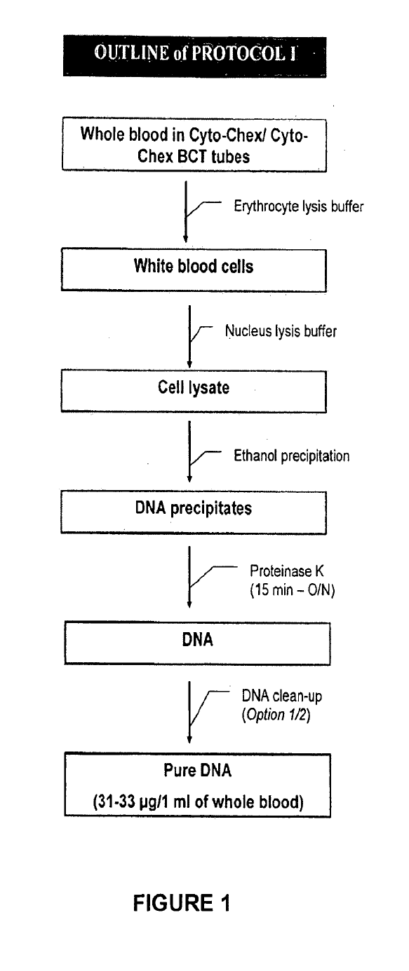

FIG. 2 is a flow diagram illustrating an example protocol for a DNA isolation method.

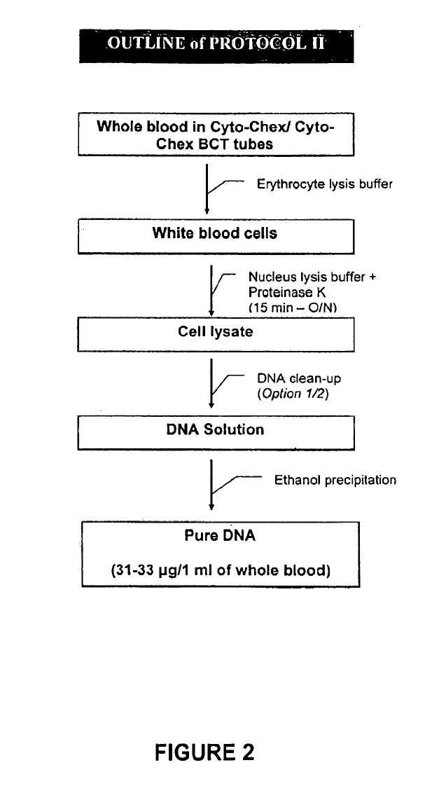

### DETAILED DESCRIPTION

In general, the invention herein contemplates a method of improved DNA isolation which includes initial fixing of a blood or tissue sample, proper storage of the sample in an appropriate device, and processing the blood or tissue sample through a number of lysing and protein removal steps to arrive at isolated DNA.

The present invention provides a method for the isolation of nucleic acids. The nucleic acid may be DNA or RNA or any combination thereof. In one preferred embodiment, nucleic acid is nuclear DNA or mitochondrial DNA. The samples from which the nucleic acids may be isolated include any biological sample including whole blood. The method disclosed herein allows for the efficient isolation of DNA and RNA samples with little to no shearing and few contaminants through the initial fixing of a tissue or blood sample.

The process for improved DNA isolation begins by contacting a blood or tissue sample with a fixative to maintain the integrity of the components within the sample, primarily the integrity of those components containing DNA. Fixatives that may be used include, but are not limited to, diazolidinyl urea, imidazolidinyl urea, dimethoylol-5,5-dimethylhydantoin, dimethylol urea, 2-bromo-2.-nitropropane-1,3-diol, oxazolidines, sodium hydroxymethyl glycinate, 5-hydroxymethoxymethyl-1-1aza-3,7-dioxabicyclo[3.3.0]octane, 5-hydroxymethyl-1-1aza-3,7dioxabicyclo[3.3.0]octane, 5-hydroxypoly[methyleneoxy]methyl-1-1aza-3,7dioxabi cyclo[3.3.0]octane, quaternary adamantine and combinations thereof.

The initial fixing of the tissue or blood sample has the effect of preserving the nucleic acids within the cells. The fixing step will also provide a sample with a longer shelf life. In a preferred embodiment, the fixative solutions comprise an active agent in solution. Suitable solvents include water, saline, dimethylsulfoxide, alcohol and mixtures thereof. Preferably, the fixative solution comprises diazolidinyl urea (Du) and/or imidazolidinyl urea (IDU) in a buffered salt solution. In a highly preferred embodiment, the fixative solution further comprises polyethylene glycol and EDTA.

The preferred solvent depends upon the tissue or cells being fixed. For example, where large pieces of tissue are being fixed, it is preferable to use an alcohol solvent since the alcohol solvents increase penetration. Preferably, the alcohol solvents comprise one or more alkanols and/or polyols.

The amount of an active agent used to fix a tissue or blood sample is generally about 10 to about 200 grams per liter. In a preferred embodiment the fixative solutions comprise about 4 to about 6 grams of IDU per 100 ml of buffered salt solution and/or about 1 to about 20 grams of Du per 100 ml of buffered salt solution.

In a preferred embodiment, the initial fixing step can occur within a specialized device, wherein the fixative agent is already present in the device prior to addition of the tissue or blood sample. More preferably, the device is an evacuated collection container, usually a tube. The tube is preferably made of a transparent material that will also resist adherence of the cells within a given sample. Most preferably, the tube further includes an anticoagulant agent and a fixative agent including but not limited to those disclosed above. The tube may also optionally include polyarcylic acid or another suitable acid. Preferably, the compounds included in the tube are in an amount sufficient to preserve the cells' morphology and nucleic acids without significant dilution of the cells. In another preferred embodiment, blood is fixed simultaneously as it is drawn into the specialized tube. The tube may also be coated with a protective coating.

In one preferred embodiment, the step of fixing allows the blood or tissue sample to be stored for a period of time prior to the DNA isolation process. More preferably, a blood or tissue sample may be drawn at one location and fixed and later transported to a different remote location for the DNA isolation process. In one preferred embodiment, the results from the DNA isolation process are analyzed at the remote location and the resulting diagnostic information is reported to the site of the original blood draw. In another preferred embodiment, the results from the DNA isolation process may be sent from the remote location and analyzed at a third location or alternatively the results may be sent back to the site of the initial blood draw and analyzed there. The resulting diagnostic information may then be sent to a third location or back to the remote location or the site of the initial blood draw.

In one preferred embodiment, the fixing step allows for the DNA isolation process to take place about 3 days after fixing. In another preferred embodiment, the DNA isolation process may take place about 24 hours after fixing. Preferably, the DNA isolation process may take place about 12 hours after fixing. More preferably, DNA isolation process may take place about 6 hours after fixing

At any time after the initial fixing of the tissue or blood sample, the sample can be treated to isolate the nucleic acids located within the sample cells. Preferably, if the DNA is being extracted from blood cells, it is first necessary to break the cell membranes or lyse the blood cells in order to access the nucleic acids within the cell nuclei or mitochondria. Post-lysing and throughout the isolation process it is important to constantly remove all unwanted materials and/or contaminants from the sample. Preferably, this is done by centrifuging the sample for any where from 2 minutes to 20 minutes and discarding the supernatant. Preferably, the lysing step followed by centrifuging is repeated a number of times in an effort to remove as many contaminants as possible.

One preferred embodiment of the present invention is a method for isolating DNA from whole blood. The method can be performed on a single sample or on a multitude of samples in a multi-well plate. The method includes fixing the starting material as previously discussed, then mixing the fixed sample with a lysing substance to break the red blood cells. The sample is then centrifuged and the supernatant is discarded. The lysing and centrifuging steps are repeated until a visual inspection indicates that contaminants have been minimized. An appropriate concentration of salt and alcohol is added to precipitate DNA containing material. Proteinase K or a similar enzyme is then added to release DNA from cross-linked proteins in the DNA containing material. An organic compound such as a phenol derivative or the like is then added to remove any remaining protein contaminants. Any protein contaminants that still remain can be removed by adding additional amounts of an organic compound such as a phenol derivative or the like. After centrifugation, ethanol is added and the sample is centrifuged again. Any remaining liquid is removed from the sample and only the DNA will remain. In one preferred embodiment, the finished product of isolated DNA is contacted with a buffer.

In a preferred embodiment, the cell lysis step is performed by a buffer, preferably an erythrocyte lysis buffer which may contain NH₄Cl, NH₃HCO₃, EDTA, sodium dodecyl sulfate, NaOH, sodium citrate, sodium acetate, citric acid, HCl, cacodylic acid sodium salt, sodium dihydrogen phosphate, disodium hydrogen phosphate, imidazole, triethenolamine hydrochloride, tris-HCl, or combinations thereof.

The cell lysis step may also be performed by a nucleus lysis buffer which may contain tris-HCl, EDTA, SDS, NH₄Cl, NH₃HCO₃, sodium dodecyl sulfate, NaOH, LiCl, sodium citrate, sodium acetate, citric acid, HCl, cacodylic acid sodium salt, sodium dihydrogen phosphate, disodium hydrogen phosphate, imidazole, triethenolamine hydrochloride, polysorbate, octyl phenol ethoxylate, or combinations thereof.

Incubation may occur on ice or at any temperature between −30° C. and 70° C. Preferably, a sample should be incubated at about −20° C. after all lysis steps have been completed. In one preferred embodiment, a sample is incubated in a water bath at about 50-65° C. after addition of proteinase K. Preferably, centrifugation occurs at speeds of about 500 to about 15,000 rpm. More preferably, centrifugation occurs at about 1,000 to 13,000 rpm. In one preferred embodiment, centrifugation is performed at about 1-20° C. More preferably, centrifugation is performed at about 4-9° C.

It will be appreciated from the teachings herein including the teachings of U.S. Provisional Application Ser. No. 60/974,115, filed Sep. 21, 2007 (see e.g. Appendices I & II, incorporated by reference) that the following are illustrations of how the present invention may be practiced.

### Example 1

Mix 1 ml of whole blood with 5 ml of erythrocyte lysis buffer in a 15 ml centrifuge tube. Vortex briefly and incubate for 10 to 20 minutes on ice to lyse the red blood cells. Centrifuge at 1000 rpm for 10 minutes at 4-9° C. and discard the supernatant. Add 2 ml of erythrocyte lysis buffer to cell pellet. Re-suspend the cells by vortexing briefly at high speed. Centrifuge at 1000 rpm for 10 minutes at 4-9° C. and discard supernatant. It is important to remove the supernatant as much as possible to avoid incomplete lysis of white blood cells. If desired, the process can be stopped at this point and the cell pellet can be maintained at −80° C. for many months. To proceed, prepare a white blood cell lysis buffer by adding β-mercaptoethanol to nucleus lysis buffer in a 1:100 dilution ratio. Mix well by inverting. Add the white blood cell lysis buffer to the cell pellet and vortex until no cell clumps are visible. Transfer the cell lysate to a clean microcentrifuge tube. Add 1/10 volume ( 1/10 of cell lysate) of 5M NaCl to the cell lysate and mix well by inverting. Add 1 volume (equal volume of cell lysate) of 100% isopropanol to the cell lysate and mix well by inverting. Incubate at −20° C. for a minimum of 20 minutes. Again, the process can be stopped at this point as the DNA is considered stable. To proceed, centrifuge at 4° C. and 13,000 rpm for 20-30 minutes. Pour off the supernatant and discard. Add 1 ml 70% ethanol to the pellet, vortex for 1 to 10 seconds and centrifuge at 4° C. and 13,000 rpm for 10 minutes. Pour off the supernatant and discard. Repeat the addition of ethanol and centrifuging. Drain the microcentrifuge tube and allow DNA pellet to air dry in the open tube. Add 200 μl TE buffer to the tube. Add proteinase K (100 μg/ml) to the DNA solution, mix gently, and incubate at 56° C. for about 0.5 to 12 hours.

### Example 2

Repeat all steps of Example 1. Continue by adding an equal volume of phenol:chloroform:isoamyl alcohol (25:24:1 saturated with 10 mM Tris, pH 8.0 or 1 mM EDTA) to DNA solution. Vortex for 10 seconds and centrifuge at 12,000 rpm at room temperature for 5 minutes. Take the aqueous phase containing the DNA and transfer to a new tube. Add 1/10 volume ( 1/10 of cell lysate) of 5M NaCl to the cell lysate and mix well by inverting. Add 1 volume (equal volume of cell lysate) of 100% isopropanol to the cell lysate and mix well by inverting. Incubate at −20° C. for a minimum of 30 minutes. Again, the process can be stopped at this point as the DNA is considered stable. To proceed, centrifuge at 4° C. and 13,000 rpm for 20-30 minutes. Pour off the supernatant and discard. Add 1 ml 70% ethanol to the pellet, vortex for 10 seconds and centrifuge at 4° C. and 13,000 rpm for 10 minutes. Pour off the supernatant and discard. Repeat the addition of ethanol and centrifuging. Drain the microcentrifuge tube and allow DNA pellet to air dry in the open tube. Add 200 μl TE buffer to the tube.

### Example 3

Repeat all steps of Example 1. Add 67 μl of protein precipitation solution to DNA solution. Vortex to mix and incubate on ice for 5 minutes. Centrifuge at 13,000 rpm at room temperature for 10 minutes. Remove the supernatant to a clean microcentrifuge tube. Add 1/10 volume ( 1/10 of cell lysate) of 5M NaCl to the cell lysate and mix well by inverting. Add 1 volume (equal volume of cell lysate) of 100% isopropanol to the cell lysate and mix well by inverting. Incubate at −20° C. for a minimum of 30 minutes. Again, the process can be stopped at this point as the DNA is considered stable. To proceed, centrifuge at 4° C. and 13,000 rpm for 20-30 minutes. Pour off the supernatant and discard. Add 1 ml 70% ethanol to the pellet, vortex for 10 seconds and centrifuge at 4° C. and 13,000 rpm for 10 minutes. Pour off the supernatant and discard. Repeat the addition of ethanol and centrifuging. Drain the microcentrifuge tube and allow DNA pellet to air dry in the open tube. Add 200 μl TE buffer to the tube.

### Example 4

Mix 1 volume of whole blood with 5 volumes of erythrocyte lysis buffer in a centrifuge tube. Vortex briefly and incubate for 10 to 20 minutes on ice to lyse the red blood cells. Centrifuge at 1000 rpm for 10 minutes at 4-9° C. and discard the supernatant. Add 2 volumes of erythrocyte lysis buffer to the cell pellet, re-suspend the cells by vortexing at high speed. Centrifuge at 1000 rpm for 10 minutes at 4-9° C. and discard supernatant. Prepare white blood cell lysis buffer by adding β-mercaptoethanol to nucleus lysis buffer in a 1:100 dilution ratio and proteinase K (100 g/μl). Add white blood cell lysis buffer to the cell pellet, vortex and incubate cell lysate mixture at 50-65° C. for 15 minutes to overnight. Cool the lysate to room temperature for 2 minutes and add 1/10 volume (equal volume of cell lysate) of 5M NaCl to the cell lysate and mix well by inverting. Add 1 volume (equal volume of cell lysate) of 100% isopropanol to the cell lysate and mix well by inverting. Incubate at −20° C. for a minimum of 30 minutes. Again, the process can be stopped at this point as the DNA is considered stable. To proceed, centrifuge at 4° C. and 13,000 rpm for 20-30 minutes. Pour off and discard the supernatant. Add 1 ml 70% ethanol to the pellet and vortex for 1 second. Centrifuge at 4° C. and 13,000 rpm for 10 minutes. Pour off and discard the supernatant. Add another 1 ml 70% ethanol to the pellet and vortex for 1 second. Centrifuge at 4° C. and 13,000 rpm for 10 minutes. Pour off and discard the supernatant. Drain the tube and allow the DNA pellet to air dry in an open tube. Add 200 μl TE buffer to the tube.

It will be appreciated that concentrates or dilutions of the amounts recited herein may be employed. In general, the relative proportions of the ingredients recited will remain the same. Thus, by way of example, if the teachings call for 30 parts by weight of a Component A, and 10 parts by weight of a Component B, the skilled artisan will recognize that such teachings also constitute a teaching of the use of Component A and Component B in a relative ratio of 3:1.

It will be appreciated that the above is by way of illustration only. Other ingredients may be employed in any of the compositions disclosed herein, as desired, to achieve the desired resulting characteristics. Examples of other ingredients that may be employed include antibiotics, anesthetics, antihistamines, preservatives, surfactants, antioxidants, unconjugated bile acids, mold inhibitors, nucleic acids, pH adjusters, osmolarity adjusters, or any combination thereof.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention at set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

1. A method of isolating DNA comprising:
providing a tube containing an anticoagulant agent and a fixative agent selected from the group consisting of diazolidinyl urea, imidazolidinyl urea and combinations thereof;
suspending a sample in the fixative agent;
contacting the sample with an erythrocyte lysis buffer;
contacting the sample with a nucleus lysis buffer; and
contacting the sample with proteinase K and alcohol.

2-4. (canceled)

5. The method of claim 1, wherein the erythrocyte lysis buffer includes ammonium chloride, ammonium bicarbonate, and a chelating agent.

6. The method of claim 5, wherein the chelating agent is EDTA.

7. The method of claim 1, wherein the nucleus lysis buffer includes ingredients selected from the group consisting of a chelating agent, a buffer, an anionic surfactant, a polysorbate surfactant, a non-ionic surfactant, and a chaotrope.

8. The method of claim 1, wherein the nucleus lysis buffer includes a buffer, a chelating agent, a polysorbate surfactant, a non-ionic surfactant and a chaotrope.

9. The method of claim 1, wherein the nucleus lysis buffer includes a buffer, a chelating agent and an anionic surfactant.

10. The method of claim 8, wherein the chelating agent is EDTA.

11. The method of claim 8, wherein the buffer is tris-HCL.

12. The method of claim 8, wherein the chelating agent is EDTA and the anionic surfactant is sodium dodecyl sulfate.

13. The method of claim 1, wherein one or more samples are extracted from one or more patients at a sample extraction site.

14. The method of claim 1, wherein the fixative agent allows for the sample to be transferred to a remote location prior to a cell lysis processing step.

15. The method of claim 14, wherein isolated DNA is analyzed and resulting data is provided to the sample extraction site, the remote location, an additional location, or any combination thereof.

16. The method of claim 1, wherein prior to contact with the sample, the fixative agent is present at a concentration of about 10 to about 200 grams per liter.

17. The method of claim 1, wherein prior to contact with the sample, the fixative concentration is about 1 to about 20 grams per 100 ml of a fixative solution.

18. The method of claim 1, wherein prior to contact with the sample, the fixative concentration is about 4 to about 6 grams per 100 ml of a fixative solution.

19. The method of claim 1, wherein the sample includes RNA.

20. The method of claim 1, wherein the sample includes cell-free DNA.

21. The method of claim 20, wherein the cell-free DNA is fetal DNA.

22. The method of claim 19, wherein the RNA is cell-free RNA.

23. The method of claim 22, wherein the cell-free RNA is mRNA.

24. The method of claim 1, wherein the sample includes cellular DNA.

25. The method of claim 1, wherein the sample includes cellular RNA.

26. The method of claim 1, wherein the fixative agent allows for the sample to be processed up to 3 days after initial contact with the fixative agent.

27. The method of claim 1, wherein:
the sample is contacted by the fixative agent and anticoagulant upon blood draw; and
the sample is contacted by the erythrocyte lysis buffer, nucleus lysis buffer, proteinase K, and ethanol at least 24 hours after blood draw.

28. The method of claim 1, wherein the amount of an active agent used to fix a tissue or blood sample is generally about 10 to about 200 grams per liter.

29. The method of claim 1, wherein the fixative agent comprises about 4 to about 6 grams of imidazolidinyl urea per 100 ml of buffered salt solution.

30. The method of claim 1, wherein the fixative agent comprises about 1 to about 20 grams of diazolidinyl urea per 100 ml of buffered salt solution.
